(19) United States
(12) Patent Application Publication
(10) Pub. No.: US 2023/0190240 A1
(43) Pub. Date: Jun. 22, 2023

(54) PASSIVE COOLING FOR MEDICAL IMAGING PROBES

(71) Applicant: GE Precision Healthcare LLC, Wauwatosa, WI (US)

(72) Inventors: Reinhard Wimmer, Zipf (AT); Paul Schrey, Zipf (AT)

(21) Appl. No.: 17/558,930

(22) Filed: Dec. 22, 2021

(51) Int. Cl. A61B 8/00; A61B 8/12

(52) U.S. Cl. CPC A61B 8/546; A61B 8/12; A61B 8/4444; A61B 8/4483; B06B 1/0644

(57) ABSTRACT

Systems and methods are provided for passive cooling for medical imaging probes. A medical imaging probe configured for use in a medical imaging system may include an enhanced shaft that has a thermal body frame disposed within a space inside the shaft; and a thermal tube disposed within an interior surface of the shaft. The thermal body frame may be connected at a cap end of the shaft with a transducer. The thermal body frame and the thermal tube configured for thermal conduction, and the thermal body frame and the thermal tube are arranged such that they are in contact with one another to facilitate transfer of heat from the transducer to an exterior surface of the shaft.

### FIELD

Aspects of the present disclosure relate to medical imaging solutions. More specifically, certain embodiments relate to methods and systems for passive cooling for medical imaging probes.

### BACKGROUND

Various medical imaging techniques may be used, such as in imaging organs and soft tissues in a human body. Examples of medical imaging techniques include ultrasound imaging, computed tomography (CT) scans, magnetic resonance imaging (MRI), etc. The manner by which images are generated during medical imaging depends on the particular technique.

For example, ultrasound imaging uses real time, non-invasive high frequency sound waves to produce ultrasound images, typically of organs, tissues, objects (e.g., fetus) inside the human body. Images produced or generated during medical imaging may be two-dimensional (2D), three-dimensional (3D), and/or four-dimensional (4D) images (essentially real-time/continuous 3D images). During medical imaging, imaging datasets (including, e.g., volumetric imaging datasets during 3D/4D imaging) are acquired and used in generating and rendering corresponding images (e.g., via a display) in real-time.

In some instances, medical imaging systems may be used to conduct particular types of examination. For example, in some instances, medical imaging systems may be used in endocavity based examinations. In some instances, certain components of medical imaging systems, such as the medical imaging probes, may pose certain challenges, particularly in conjunction with certain types of examinations, and conventional and traditional approaches may not sufficiently address or overcome these challenges.

Further limitations and disadvantages of conventional and traditional approaches will become apparent to one of skill in the art, through comparison of such systems with some aspects of the present disclosure, as set forth in the remainder of the present application with reference to the drawings.

### BRIEF SUMMARY

System and methods are provided for passive cooling for medical imaging probes, substantially as shown in and/or described in connection with at least one of the figures, as set forth more completely in the claims.

These and other advantages, aspects and novel features of the present disclosure, as well as details of one or more illustrated example embodiments thereof, will be more fully understood from the following description and drawings.

### BRIEF DESCRIPTION OF THE DRAWINGS

FIG. 1 is a block diagram illustrating an example medical imaging arrangement.

FIG. 2 is a plot diagram illustrating comparisons between an example medical imaging probe with piezoelectric (PZT) ceramic transducer and an example medical imaging probe with single crystal transducer with respect to temperature, mechanical index (MI), and voltage related performance.

FIGS. 3A-3C are block diagrams illustrating an example medical imaging probe with passive cooling.

FIG. 4 is a block diagram illustrating an example test setup for examining temperature rise in a medical imaging probe with passive cooling.

FIGS. 5A and 5B are plot diagrams illustrating results of example simulation and real tests for examining temperature rise in a medical imaging probe with passive cooling.

FIGS. 6A and 6B are plot diagrams illustrating comparisons between medical imaging probes with and without thermal improvement, using both simulation and real (measurement-based) tests.

FIG. 7 is a plot diagram illustrating net gain in an example medical imaging probe with passive cooling.

### DETAILED DESCRIPTION

Certain implementations in accordance with the present disclosure may be directed to passive cooling for medical imaging probes. In particular, the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” as used in the context of ultrasound imaging is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

In addition, as used herein, the phrase “pixel” also includes embodiments where the data is represented by a “voxel.” Thus, both the terms “pixel” and “voxel” may be used interchangeably throughout this document.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC, or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. In addition, forming of images may be performed using channel combinations that may originate from more than one transmit event (e.g., synthetic aperture techniques).

In various embodiments, processing to form images is performed in software, firmware, hardware, or a combination thereof. The processing may include use of beamforming.

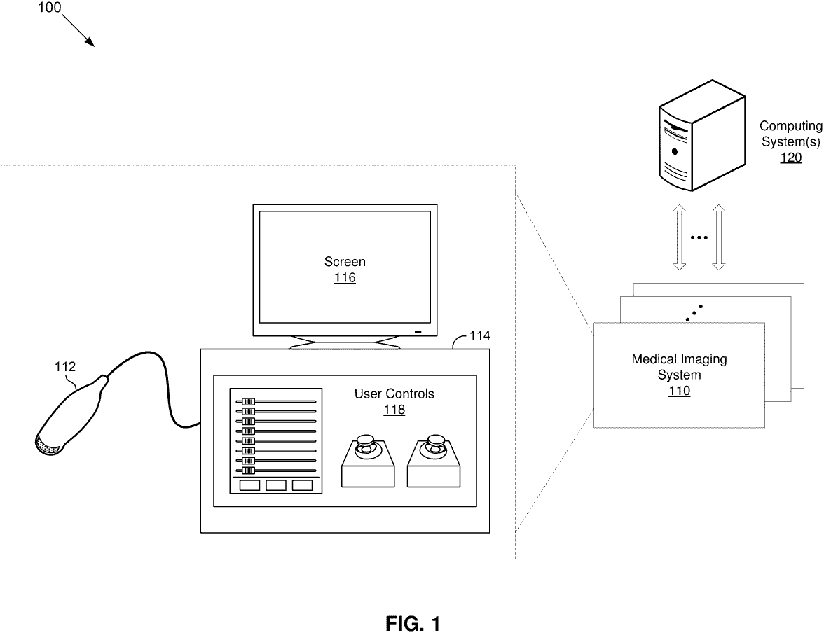

FIG. 1 is a block diagram illustrating an example medical imaging arrangement. Shown in FIG. 1 is an example medical imaging arrangement 100 that comprises one or more medical imaging systems 110 and one or more computing systems 120. The medical imaging arrangement 100 (including various elements thereof) may be configured to support medical imaging and solutions associated therewith.

The medical imaging system 110 comprise suitable hardware, software, or a combination thereof, for supporting medical imaging—that is enabling obtaining data used in generating and/or rendering images during medical imaging exams. Examples of medical imaging include ultrasound imaging, computed tomography (CT) scans, magnetic resonance imaging (MRI), etc. This may entail capturing of particular type of data, in particular manner, which may in turn be used in generating data for the images. For example, the medical imaging system 110 may be an ultrasound imaging system, configured for generating and/or rendering ultrasound images.

As shown in FIG. 1, the medical imaging system 110 may comprise a scanner device 112, which may be portable and movable, and a display/control unit 114. The scanner device 112 may be configured for generating and/or capturing particular type of imaging signals (and/or data corresponding thereto), such as by being moved over a patient’s body (or part thereof), and may comprise suitable circuitry for performing and/or supporting such functions. The scanner device 112 may be an ultrasound probe, MRI scanner, CT scanner, or any suitable imaging device. For example, where the medical imaging system 110 is an ultrasound system, the scanner device 112 may emit ultrasound signals and capture echo ultrasound images.

The display/control unit 114 may be configured for displaying images (e.g., via a screen 116). In some instances, the display/control unit 114 may further be configured for generating the displayed images, at least partly. Further, the display/control unit 114 may also support user input/output. For example, the display/control unit 114 may provide (e.g., via the screen 116), in addition to the images, user feedback (e.g., information relating to the system, functions thereof, settings thereof, etc.). The display/control unit 114 may also support user input (e.g., via user controls 118), such as to allow controlling of the medical imaging. The user input may be directed to controlling display of images, selecting settings, specifying user preferences, requesting feedback, etc.

In some implementations, the medical imaging arrangement 100 may also incorporate additional and dedicated computing resources, such as the one or more computing systems 120. In this regard, each computing system 120 may comprise suitable circuitry, interfaces, logic, and/or code for processing, storing, and/or communication data. The computing system 120 may be dedicated equipment configured particularly for use in conjunction with medical imaging, or it may be a general purpose computing system (e.g., personal computer, server, etc.) set up and/or configured to perform the operations described hereinafter with respect to the computing system 120. The computing system 120 may be configured to support operations of the medical imaging systems 110, as described below. In this regard, various functions and/or operations may be offloaded from the imaging systems. This may be done to streamline and/or centralize certain aspects of the processing, to reduce cost-e.g., by obviating the need to increase processing resources in the imaging systems.

The computing systems 120 may be set up and/or arranged for use in different ways. For example, in some implementations a single computing system 120 may be used; in other implementations multiple computing systems 120, either configured to work together (e.g., based on distributed-processing configuration), or separately, with each computing system 120 being configured to handle particular aspects and/or functions, and/or to process data only for particular medical imaging systems 110. Further, in some implementations, the computing systems 120 may be local (e.g., co-located with one or more medical imaging systems 110, such within the same facility and/or same local network); in other implementations, the computing systems 120 may be remote and thus can only be accessed via remote connections (e.g., via the Internet or other available remote access techniques). In a particular implementation, the computing systems 120 may be configured in cloud-based manner, and may be accessed and/or used in substantially similar way that other cloud-based systems are accessed and used.

Once data is generated and/or configured in the computing system 120, the data may be copied and/or loaded into the medical imaging systems 110. This may be done in different ways. For example, the data may be loaded via directed connections or links between the medical imaging systems 110 and the computing system 120. In this regard, communications between the different elements in the medical imaging arrangement 100 may be done using available wired and/or wireless connections, and/or in accordance any suitable communication (and/or networking) standards or protocols. Alternatively, or additionally, the data may be loaded into the medical imaging systems 110 indirectly. For example, the data may be stored into suitable machine readable media (e.g., flash card, etc.), which are then used to load the data into the medical imaging systems 110 (on-site, such as by users of the systems (e.g., imaging clinicians) or authorized personnel), or the data may be downloaded into local communication-capable electronic devices (e.g., laptops, etc.), which are then used on-site (e.g., by users of the systems or authorized personnel) to upload the data into the medical imaging systems 110, via direct connections (e.g., USB connector, etc.).

In operation, the medical imaging system 110 may be used in generating and presenting (e.g., rendering or displaying) images during medical exams, and/or in supporting user input/output in conjunction therewith. The images may be 2D, 3D, and/or 4D images. The particular operations or functions performed in the medical imaging system 110 to facilitate the generating and/or presenting of images depends on the type of system—that is, the manner by which the data corresponding to the images is obtained and/or generated. For example, in ultrasound imaging, the data is based on emitted and echo ultrasound signals. In computed tomography (CT) scans based imaging, the data is based on emitted and captured x-rays signals.

In various implementations in accordance with the present disclosure, medical imaging systems and/or architectures (e.g., the medical imaging system 110 and/or the medical imaging arrangement 100 as a whole) may be configured to support implementing and utilizing medical imaging probes, particularly endocavity probes, with thermal improvement, particularly passive cooling. In this regard, as used in this disclosure, “new probes” refer to medical imaging probes incorporating thermal features (particularly passive cooling) in accordance with the disclosure whereas “legacy probes” refer to medical imaging probes not incorporating such thermal features. Thus as used herein, legacy probes and new probes may be probes that are otherwise similar (including using the same transducer) except for the use (or not) of thermal improvement features, as described in the disclosure, therein.

Incorporating thermal improvement features may be desirable. In this regard, because medical imaging probes come into contact with patients’ bodies in the course of medical imaging operations, thermal characteristics (and challenges relating thereto) of these probes may be pertinent and important consideration. The thermal characteristics may include or pertain to, for example, the temperature of the probes and any changes thereof during medical imaging operations. For example, heat may be generated in medical imaging probes during operation thereof, such as in any transducer(s) incorporated into the probe as transducer(s) are emitting imaging related signals, and/or when processing components (if any) are operating—e.g., in conjunction with processing transmitted signals and captured echo signals.

Because, as noted, medical imaging probes typically may be in contact with patients’ bodies, improving thermal performance of the probes is desirable as it is important the temperature of the probes is controlled to ensure that it remains within acceptable range(s). This is may be particularly the case in endocavity medical imaging probes/operations, as the probes (or at least a portion thereof) may be inserted within a body cavity (e.g., vaginal canal), as sensitivity to temperature typically would be higher in such cavity compared to external skin. With newer designs, however, controlling temperature (and particularly with respect to dissipation of heat) may be even more important of a consideration. In this regard, due to the more efficient design (e.g., single crystal technology than piezoelectric (PZT) ceramic transducer technology), higher temperatures may occur at the cap of probe. Such probes incorporating newer designs may have high margin regarding mechanical index (MI) and also maximum applied voltage. A big limiting factor is the maximum allowed temperature rising, however.

Accordingly, medical imaging probes may incorporate features for controlling and enhancing thermal performance of the probes. In particular, medical imaging probes based on the present disclosure may incorporate passive cooling measures—that is, for controlling the temperature and heat dissipation of the probes. The passive cooling measures may be particularly incorporated into the shafts of the probes. In legacy probes the shaft may not provide such cooling functions. For example, in various example legacy probes, the probe may be completely filled with oil or other similar sustenance which may have nearly the same thermal conductivity as air. In other words, example legacy probes may be well thermally insulated. Such thermal insulation may result in adverse thermal performance of the probe as a whole, however. In this, regard, when heat generated in the cap area of the probe is not allowed to dissipate through the shaft (that is away from the contact area with the patient’s body), the heat would transfer to the patient instead. Thus, to maintain acceptable thermal performance, the operational performance of the probe will need to be actively adjusted, which may result in reduced imaging performance.

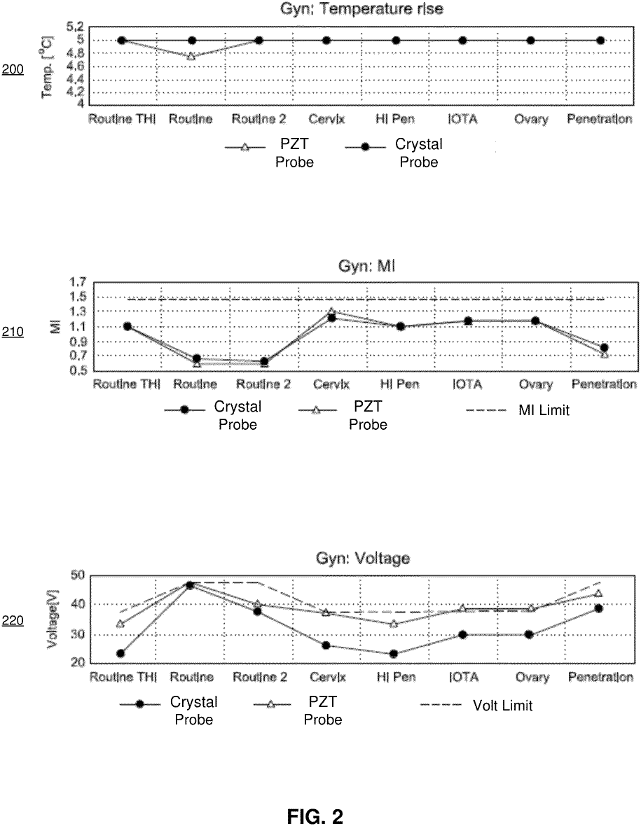

By using passive cooling, such as by incorporating passive cooling measures within the shaft, at least some of the heat may be dissipated away from the cap, thus allowing for use of more optimal (imaging wise) operation parameters of the active components of the probe. In example new probes a new shaft design may be used, with no oil (or similar substance) used in the shaft (optionally with some oil (or similar substance) in the cap area), which additionally provides additional improvement in terms of weight reduction; rather, with the shaft incorporating a design that allows for dissipating heat via the shaft surface. In this regard, shaft incorporating such enhanced design may conduct the heat energy away from the cap/transducer back to the shaft and at the end it is dissipated via the shaft surface. Example implementations and details related thereto are described with respect to FIGS. 2-7.

FIG. 2 is a plot diagram illustrating comparisons between an example medical imaging probe with piezoelectric (PZT) ceramic transducer and an example medical imaging probe with single crystal transducer with respect to temperature, mechanical index (MI), and voltage related performance. Shown in FIG. 2 are plots 200, 210, and 220 which illustrate, respectively, maximum temperature, Mechanical Index (MI), and voltage for medical imaging probes for different types of medical imaging tests (e.g., different types of gynecological based endocavity medical imaging testing).

In particular, plot 200 illustrates the maximum temperature that may be reached for each of the different example medical imaging based tests. In this regard, as shown in plot 200, as with a probe with PZT ceramic transducer, a probe with single crystal transducer need to meet particular thermal performance requirements—e.g., not exceed predefined maximum temperature(s). For example, for the medical imaging tests captures in the plots of FIG. 2, the maximum temperature of the probe should not exceed ~5.0° C. The thermal performance of the probes (and meeting any defined thermal requirements) may impact and/or be affected by other characteristics and/or performance parameters of the probe, and as such these other characteristics and/or performance parameters may need to be controlled or adjusted to ensure meeting the thermal (e.g., maximum temperature) requirements of the probes.

For example, because mechanical index (MI) and voltage (e.g., maximum voltage applied to its transducer) of the probe may have bearing on the thermal characteristics (and thus temperature) of the probes, these parameters may need to remain within limits that are ensure that the probes meet the thermal requirements. As shown in plots 210 and 220, the probe with single crystal transducers(s) may be able to provide equal or better performance (e.g., high margins) with respect to the mechanical index (MI) and maximum voltage compared to probe with PZT ceramic transducers(s) within the constraints of the same maximum temperature permitted for various tests in which the probes may be used. In this regard, as illustrated in plots 210 and 220, with penetration mode of operation, probes with single crystal transducers may offer high margins regarding mechanical index (MI) and the maximum voltage. The voltage may be set higher, for example, without depolarizing the crystal(s) used in the probes, such as with the help of the DC-bias implementation.

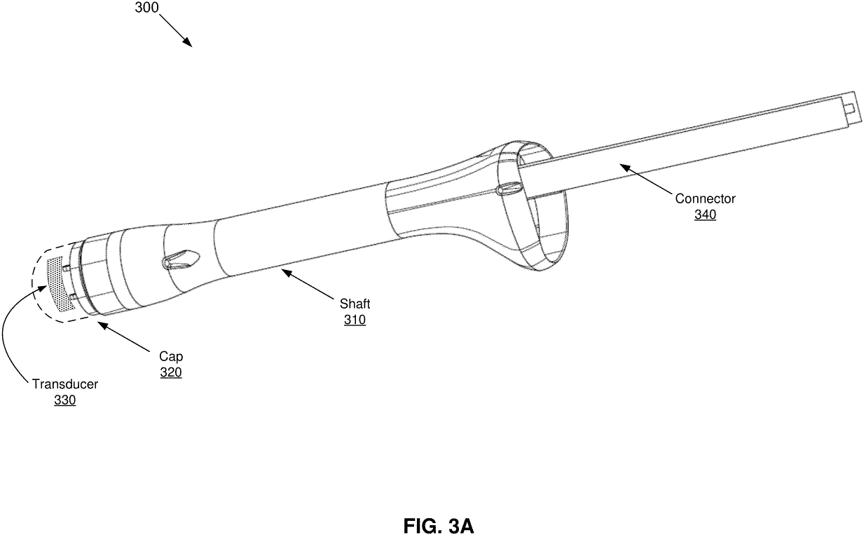

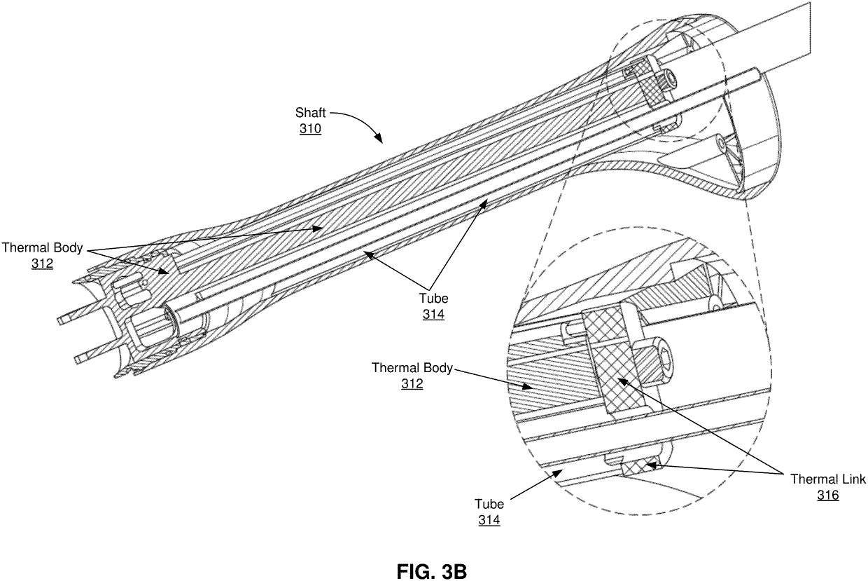

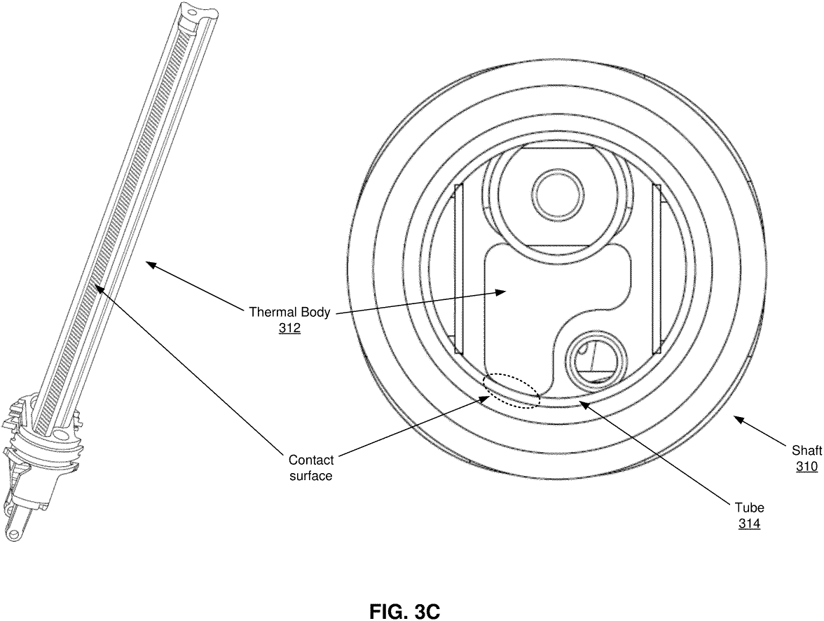

Therefore, because the primary limitation affecting operation of probes may be the maximum allowed temperature rise, lowering the temperature rise as much as possible may allow for fully utilizing performance margins in probes with single crystal transducers. This may be done by incorporating passive cooling features, which allow for enhanced dissipation of heat, and thus decreasing or slowing temperature rise in the probe. An example implementation of such probe(s) with such passive cooling is shown and described with respect to FIGS. 3A-3C.

FIGS. 3A-3C are block diagrams illustrating an example medical imaging probe with passive cooling. Shown in FIGS. 3A-3C is a medical imaging probe (or portion thereof) 300, which is implemented in accordance with the present disclosure—that is, with features for facilitating passive cooling, particularly in endocavity testing use cases.

As shown in FIG. 3A, the medical imaging probe 300 comprises a shaft 310 that incorporates various features for facilitating passive cooling during medical imaging operations. As shown in FIG. 3A, the shaft 310 has or connects to a cap (end) 320 on one side, where a transducer 330 (or transducer array, or any suitable signal transmitting/receiving component based on the medical imaging technique being used) is disposed for use in conjunction with medical imaging operations. The transducer 330 is connected to the remainder of the medical imaging system/arrangement (not shown) via a connector 340, which provide power, control, etc. to the transducer, and is also used to receive signals, data, etc. from the transducer 330.

The medical imaging probe 300 (and particularly the shaft 310 thereof) incorporate passive cooling design. In this regard, as shown in FIG. 3B, the shaft 310 may be have hollow design, with an external shell (e.g., of molded plastic or the like) with an internal thermal body (or frame) 312 within the shaft. The thermal body may provide framing and structural support to the shaft, while also providing thermal dissipation, by providing a heat dissipation path from the transducer end. For example, the thermal body 312 may comprise suitable material (e.g., metal or metallic alloy) selected for optimal performance, thermal and non-thermal—that is, with desirable thermal conductivity while also having acceptable structural strength characteristics, low weight, low cost, etc. For example, in an example implementation, the thermal body 312 may be constructed from aluminum based frame, as illustrated in FIGS. 3B and 3C.

The external shell of the shaft may comprise a thermal tube 314 which may be in contact with the thermal body 312, to facilitating heat dissipation via the surface of the shaft 310. In this regard, the thermal tube 314 may comprise suitable material (e.g., metal or metallic alloy) selected for optimal performance. For example, the thermal tube 314 may comprise a metallic (e.g., aluminum) tube. The thermal tube may be molded together with the shaft in order to reach a higher thermal conductivity.

As shown in FIG. 3C, the shaft 310 may be designed and implemented such that there may be a contact surface along the shaft 310 (or at least a portion thereof) between the thermal body 312 and the thermal tube 314. Further, in some instance, a thermal link 316 may also be used to secure the thermal body 312 while also providing additional connection to the thermal tube 314. The thermal link 316 (and/or the contact surface) may incorporate phase change thermal interface materials, for enhanced thermal performance.

Accordingly, in the example implementation illustrated in FIGS. 3A-3C, the design of the shaft 310 may ensure that heat is dissipated from the cap/transducer side (left) through the shaft.

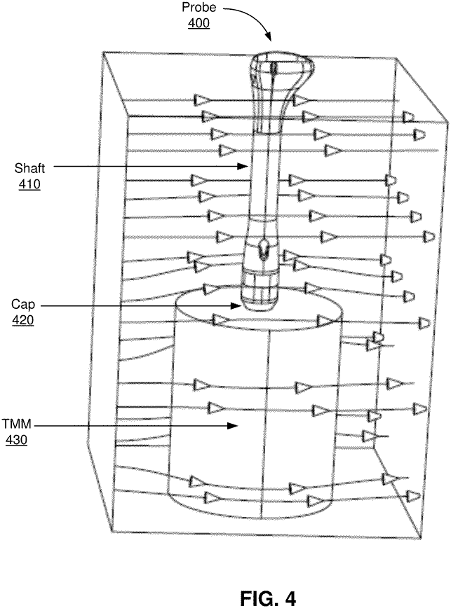

FIG. 4 is a block diagram illustrating an example test setup for examining temperature rise in a medical imaging probe with passive cooling. Shown in FIG. 4 is a test that may be used for examining temperature rise in a medical imaging probe 400 (or portion thereof-namely, a shaft 410) incorporating passive cooling features as described herein. For example, the probe 400 may be similar to the probe 300, with its shaft 410 incorporating similar design as that of the shaft 310 as described with respect to FIGS. 3A-3C.

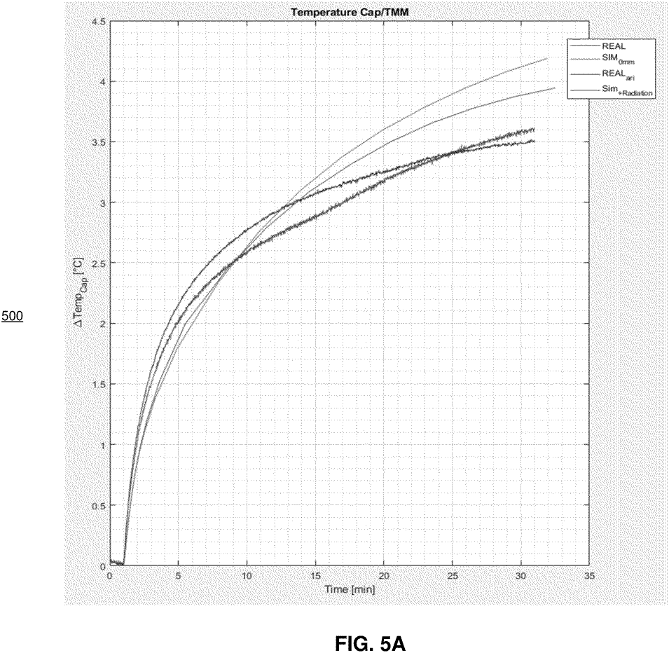

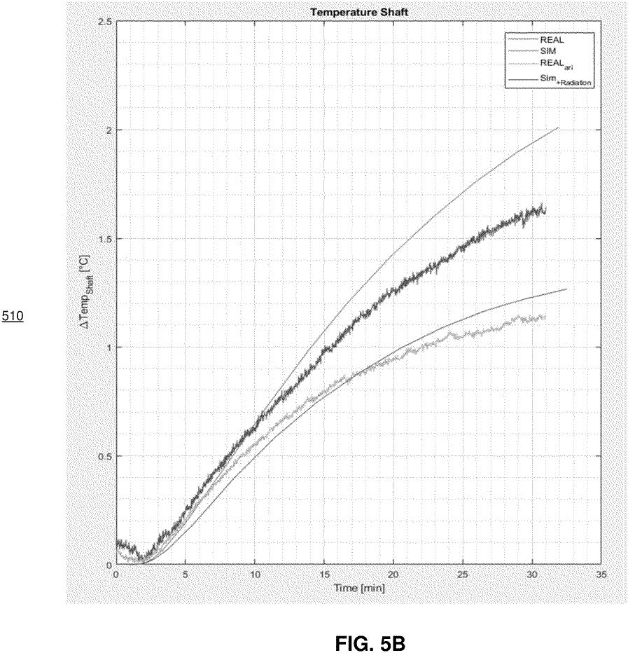

As illustrated in FIG. 4, the probe 400 may be placed on top of a tissue mimicking material (TMM) 430 (or TMM phantom), with a cap 420 of the probe 400 in contact with the TMM 430, and the shaft 410 section extending horizontally from the TMM 430. Heat may then applied within a space containing the TMM 430 and the probe 400, as illustrated in FIG. 4. The heat may be increased, with the temperature of the probe 400 (and particularly changes thereof) monitored and recorded. In this regard, the temperature may be tracked and recorded for different parts, such as at the cap/TMM contact area and in the shaft itself (or, more specifically, at the surface of the shaft at some distance from the cap/TMM contact area). Further, in some instances, radiation energy may also be included. Results of example tests (simulation and real tests) using a setup/arrangement as the one illustrated in FIG. 4 are described with respect to FIG. 5A-7.

FIGS. 5A and 5B are plot diagrams illustrating results of example simulation and real tests for examining temperature rise in a medical imaging probe with passive cooling. Shown in FIGS. 5A and 5B are plots 500 and 510

As shown in FIGS. 5A and 5B, plots 500 and 510 capture data corresponding to example results of tests (simulation and real/measurement tests) on an example medical imaging probe with passive cooling in accordance with the present disclosure, such as using the testing setup/arrangement illustrated in and described with respect to FIG. 4.

Illustrated in plots 500 and 510 are changes (specifically rise) in temperature (ΔT) in Celsius (°C) for, respectively, the cap/TMM contact area and the shaft of the probe, as a function of time (in minutes). As plots 500 and 510 show, the simulation test shows almost the same temperature rise as the measurement results based on real test(s). If radiation energy is also included, there may be some deviation in the maximum temperature between simulation and measurement. For example, the test data used in generating plots 500 and 510 show deviation in the maximum temperature between simulation and measurement of approx. 0.3° C. In this regard, the transient data shows a certain deviation, since the specific heat capacity is not exactly known for all materials.

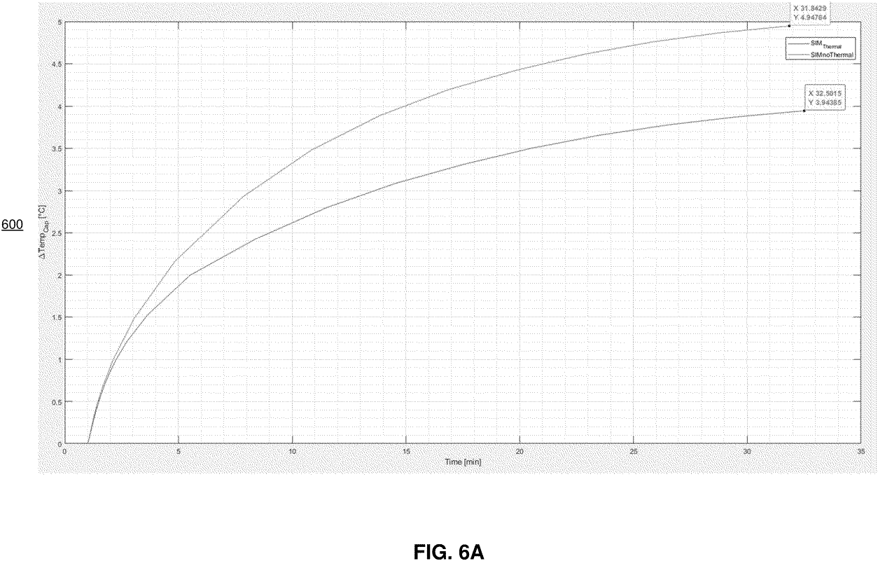

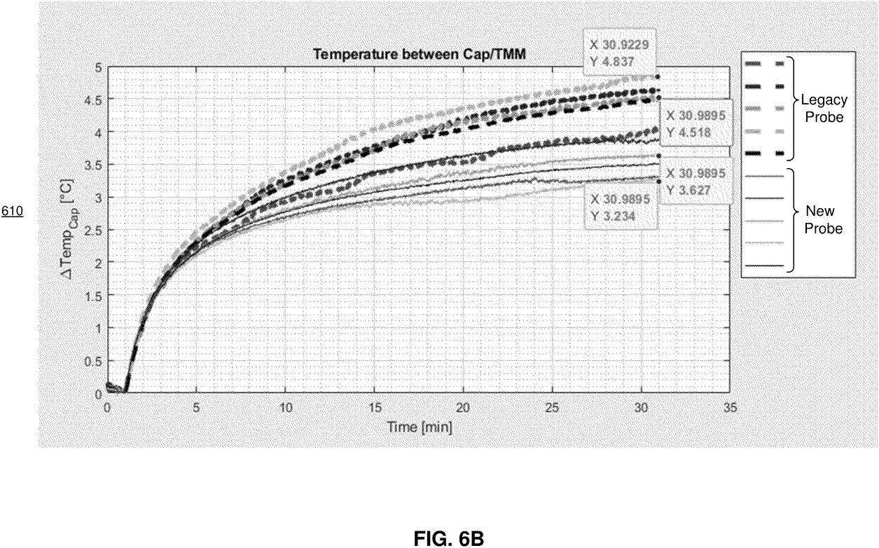

FIGS. 6A and 6B are plot diagrams illustrating comparisons between medical imaging probes with and without thermal improvement, using both simulation and real (measurement-based) tests. Shown in FIGS. 6A and 6B are plots 600 and 610.

As shown in FIGS. 6A and 6B, plots 600 and 610 capture data corresponding to example results for, respectively, simulation based test and real/measurement based test(s), such as using the testing setup/arrangement illustrated in and described with respect to FIG. 4, performed using example medical imaging probes with no thermal improvement (that is, legacy probes) and medical imaging probes with thermal improvement (e.g., passive cooling) in accordance with the present disclosure, with the testing done under otherwise similar operating conditions for both types of probes. In this regard, as noted legacy probes and new probes may be probes that are otherwise similar (including using the same transducer) except for the use (or not) of thermal improvement features.

Illustrated in plots 600 and 610 are changes (specifically rise) in temperature (ΔT) in Celsius (°C) for the cap/TMM contact area of each tested probe, as a function of time (in minutes). As plots 600 and 610 show, medical imaging probes with thermal improvement (e.g., passive cooling) show clear improvement compared to medical imaging probes without thermal improvement. For example, as illustrated in plot 600, the results of simulation testing show that with time, medical imaging probes without thermal improvement have temperature rise that is higher (ΔT[°C] ~1° C.). Similarly, as illustrated in plot 610, the measurement results of real tests (multiple runs) show that with time, medical imaging probes without thermal improvement have temperature rise that is higher (ΔT[°C] ~0.9° C.-1.5° C.).

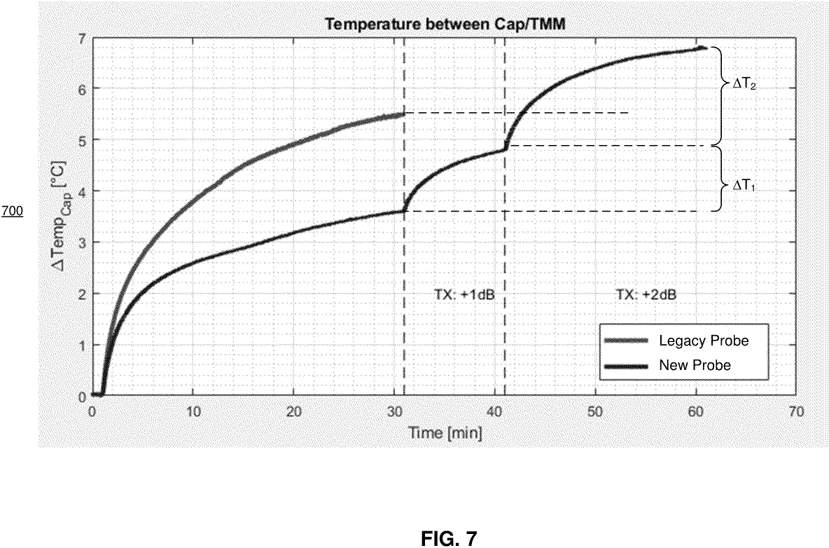

FIG. 7 is a plot diagram illustrating net gain in an example medical imaging probe with passive cooling. Shown in FIG. 7 is plot 700.

As shown in FIG. 7, plot 700 capture data corresponding to example results of testing using a legacy probe and a new probe-that is, using, respectively, an example medical imaging probe with no thermal improvement and an example medical imaging with thermal improvement (e.g., passive cooling)—with power adjustments during the test. In this regard, as noted legacy probes and new probes may be probes that are otherwise similar (including using the same transducer) except for the use (or not) of thermal improvement features.

Illustrated in plot 700 are changes (specifically rise) in temperature (ΔT) in Celsius (°C) for the cap/TMM contact area of each tested probe (the legacy probe and the new probe), as a function of time (in minutes). As shown in plot 700, power applied to the new probe may be increased (e.g., by increasing voltage) because the rise of temperature is lower.

For example, power may be initially increased by +1 dB (once the temperature at cap/TMM contact area starts to flatten out). This +1 dB increase in power may result in an increase in temperature, which may be still be small enough that the probe is below its maximum temperature. For example, as shown in plot 700, the +1 dB power increase may result in a temperature increase (ΔT) of -1.2° C. Thus, power may be increase again by another 1 dB (for total of +2 dB increase over initial power).

This second power increase may result in further increase in temperature at the cap/TMM contact area. For example, as shown in plot 700, the +2 dB power increase may result in a further temperature increase (ΔT) of -2° C. (from the point when the second power increase is applied). This may exceed the desired maximum temperature rise.

Nonetheless, power may to be increased by approx. +1.5 dB before the temperature of the new probe may reach the same maximum temperature as the probe without thermal improvement. Accordingly, plot 700 illustrates, new probes may be operated at higher power while having the same maximum temperature as legacy probes.

Accordingly, implementations based on the present disclosure allow for enhanced thermal improvement compared to legacy solutions, by offering unique transfer of heat from the transducer to the outer surface of the shaft of the probe. In an example implementation, the shaft may be molded around a thermal tube (e.g., aluminum tube) in order to get a higher thermal conductivity. The contact surface between thermal body and tube will be connected via phase change thermal interface material. This idea could also be applied to other probes such as abdominal probes. Nonetheless, even in implementations without molded thermal tubes may yield enhanced thermal performance. The new probes may still be bound by the same maximum temperature rise used with legacy probes, but the new probes larger margins in terms of other characteristics and operation parameters such as mechanical index (MI) and maximum voltage. The new probes may allow for taking advantage of newer design, such as use of single crystal. In this regard, thermal design in accordance with the present disclosure may allow for enhanced dissipation of heat, thus relieving temperature hotspots on the cap, which allows for exploiting the full potential of the single crystal. The enhanced thermal performance allow for improved performance (e.g., using higher voltage), and improving the probes as a whole (e.g., by reducing weight, by elimination of oil or other substances that may be used in existing shaft designs to make them thermally insulated. Thus, solutions in accordance with the present disclosure may allow for improved image quality, higher acoustic output at same temperature, and higher penetration depth.

An example medical imaging probe, in accordance with the present disclosure, may be configured for use in a medical imaging system, with the medical imaging probe comprising: a transducer configured to transmit and receive signals based on a medical imaging technique; and a shaft that comprises: a thermal body frame disposed within a space inside the shaft; and a thermal tube disposed within an interior surface of the shaft; wherein: the thermal body frame is connected at a cap end of the shaft with the transducer; the thermal body frame and the thermal tube configured for thermal conduction; and the thermal body frame and the thermal tube are arranged such that they are in contact with one another to facilitate transfer of heat from the transducer to an exterior surface of the shaft.

In an example embodiment, the medical imaging probe is configured for use in endocavity medical imaging examinations.

In an example embodiment, the thermal body frame comprises metal material.

In an example embodiment, the thermal tube comprises metal material.

In an example embodiment, the thermal body frame and the thermal tube comprise a same thermal conduction material.

In an example embodiment, the same thermal conduction material comprises aluminum.

In an example embodiment, the shaft further comprises a thermal link configured to secure the thermal body frame to the thermal tube, and wherein the thermal link provide further thermal connectivity between the thermal body frame and the thermal tube.

In an example embodiment, the thermal body frame is configured to allow passage of a connector within the shaft to the transducer.

In an example embodiment, the transducer comprises a single crystal transducer.

An example shaft, in accordance with the present disclosure, may be configured for use in a medical imaging probe, with the shaft comprising: a thermal body frame disposed within a space inside the shaft; and a thermal tube disposed within an interior surface of the shaft; wherein: the thermal body frame is connected at a cap end of the shaft with one or more elements that generate heat during operation of the medical imaging probe; both of the thermal body frame and the thermal tube configured for thermal conduction; and the thermal body frame and the thermal tube are arranged such that they are in contact with one another to facilitate transfer of heat away from the element and into an exterior surface of the shaft.

In an example embodiment, the medical imaging probe is configured for use in endocavity medical imaging examinations.

In an example embodiment, the thermal body frame comprises metal material.

In an example embodiment, the thermal tube comprises metal material.

In an example embodiment, the thermal body frame and the thermal tube comprise a same thermal conduction material.

In an example embodiment, the same thermal conduction material comprises aluminum.

In an example embodiment, the shaft further comprises a thermal link configured to secure the thermal body frame to the thermal tube, and wherein the thermal link provide further thermal connectivity between the thermal body frame and the thermal tube.

In an example embodiment, the thermal body frame is configured to allow passage of a connector within the shaft to the element.

As utilized herein the terms “circuits” and “circuitry” refer to physical electronic components (e.g., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” As utilized herein, the terms “block” and “module” refer to functions than can be performed by one or more circuits. As utilized herein, the term “exemplary” means serving as a non-limiting example, instance, or illustration. As utilized herein, the terms “for example” and “e.g.,” set off lists of one or more non-limiting examples, instances, or illustrations. As utilized herein, circuitry is “operable” to perform a function whenever the circuitry comprises the necessary hardware (and code, if any is necessary) to perform the function, regardless of whether performance of the function is disabled or not enabled (e.g., by some user-configurable setting, a factory trim, etc.).

Other embodiments of the invention may provide a non-transitory computer readable medium and/or storage medium, and/or a non-transitory machine readable medium and/or storage medium, having stored thereon, a machine code and/or a computer program having at least one code section executable by a machine and/or a computer, thereby causing the machine and/or computer to perform the processes as described herein.

Accordingly, the present disclosure may be realized in hardware, software, or a combination of hardware and software. The present invention may be realized in a centralized fashion in at least one computing system, or in a distributed fashion where different elements are spread across several interconnected computing systems. Any kind of computing system or other apparatus adapted for carrying out the methods described herein is suited. A typical combination of hardware and software may be a general-purpose computing system with a program or other code that, when being loaded and executed, controls the computing system such that it carries out the methods described herein. Another typical implementation may comprise an application specific integrated circuit or chip.

Various embodiments in accordance with the present disclosure may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

1. A medical imaging probe configured for use in a medical imaging system, the medical imaging probe comprising:
a transducer configured to transmit and receive signals based on a medical imaging technique; and
a shaft that comprises:
a shell
a thermal body frame disposed within a space inside the shaft; and
a thermal tube disposed within an interior surface of the shaft; wherein:
the thermal tube comprises different material than the shell;
the thermal tube is incorporated with or attached to the shaft;
the thermal body frame is connected at a cap end of the shaft with the transducer;
the thermal body frame and the thermal tube configured for thermal conduction; and
the thermal body frame and the thermal tube are arranged such that they are in contact with one another to facilitate transfer of heat from the transducer to an exterior surface of the shaft.

2. The medical imaging probe of claim 1, wherein the medical imaging probe is configured for use in endocavity medical imaging examinations.

3. The medical imaging probe of claim 1, wherein the thermal body frame comprises metal material.

4. The medical imaging probe of claim 1, wherein the thermal tube comprises metal material.

5. The medical imaging probe of claim 1, wherein the thermal body frame and the thermal tube comprise a same thermal conduction material.

6. The medical imaging probe of claim 5, wherein the same thermal conduction material comprises aluminum.

7. The medical imaging probe of claim 1, wherein the shaft further comprises a thermal link configured to secure the thermal body frame to the thermal tube, and wherein the thermal link provides further thermal connectivity between the thermal body frame and the thermal tube.

8. The medical imaging probe of claim 1, wherein the thermal body frame is configured to allow passage of a connector within the shaft to the transducer.

9. The medical imaging probe of claim 1, wherein the transducer comprises single crystal transducer.

10. A shaft configured for use in a medical imaging probe, the shaft comprising:
a shell;
a thermal body frame disposed within a space inside the shaft; and
a thermal tube disposed within an interior surface of the shaft; wherein:
the thermal tube comprises different material than the shell;
the thermal tube is incorporated with or attached to the shaft;
the thermal body frame is connected at a cap end of the shaft with one or more elements that generate heat during operation of the medical imaging probe;
both of the thermal body frame and the thermal tube configured for thermal conduction; and
the thermal body frame and the thermal tube are arranged such that they are in contact with one another to facilitate transfer of heat away from the one or more elements and into an exterior surface of the shaft.

11. The shaft of claim 10, wherein the medical imaging probe is configured for use in endocavity medical imaging examinations.

12. The shaft of claim 10, wherein the thermal body frame comprises metal material.

13. The shaft of claim 10, wherein the thermal tube comprises metal material.

14. The shaft of claim 10, wherein the thermal body frame and the thermal tube comprise a same thermal conduction material.

15. The shaft of claim 14, wherein the same thermal conduction material comprises aluminum.

16. The shaft of claim 10, wherein the shaft further comprises a thermal link configured to secure the thermal body frame to the thermal tube, and wherein the thermal link provides further thermal connectivity between the thermal body frame and the thermal tube.

17. The shaft of claim 10, wherein the thermal body frame is configured to allow passage of a connector within the shaft to the one or more elements.
